(19) United States
(12) Patent Application Publication
(10) Pub. No.: US 2021/0205204 A1
(43) Pub. Date: Jul. 8, 2021

(54) CARBOHYDRATE FRACTION ISOLATED FROM A CLARIFIED LYSATE OBTAINED FROM A BIOMASS OF BACTERIA BELONGING TO THE GENUS VITREOSCILLA SP

(71) Applicant: L'OREAL, Paris (FR)

(72) Inventors: Peiyan YU, Rabastens (FR); Lucie TOURNIER-COUTURIER, Aulnay Sous Bois (FR); Romain GARCON, Aulnay Sous Bois (FR)

(21) Appl. No.: 16/066,589

(22) PCT Filed: Dec. 23, 2016
(86) PCT No.: PCT/EP2016/082547

§ 371 Date: Jun. 27, 2018

(30) Foreign Application Priority Data
Dec. 28, 2015 (FR) 1563389

(51) Int. Cl. A61K 8/73; A61Q 19/08; A61Q 5/12

(52) U.S. Cl. CPC A61K 8/73; A61Q 5/12; A61Q 19/08

(57) ABSTRACT

The present invention relates to a carbohydrate fraction isolated from a clarified lysate obtained by thermolysis and proteolysis of a biomass of bacteria belonging to the genus Vitreoscilla sp.

### FIELD OF THE INVENTION

The present invention relates to active agents intended to be used in the field of cosmetics and/or dermatology.

More particularly, the invention relates to a carbohydrate fraction isolated from a clarified lysate obtained by thermolysis and proteolysis of a biomass of bacteria belonging to the genus Vitreoscilla sp., and to the use thereof as cosmetic and/or dermatological active agent, preferably as anti-ageing active agent.

### TECHNOLOGICAL BACKGROUND

Over time, it appears that keratin materials, such as the skin, lips, eyelashes, eyebrows, nails and hair are subject to ageing, reflected especially by a modification of their structures and their functions.

Endogenously, keratin materials are subject to attacks by superoxide ions, naturally produced during physiological cellular metabolic processes.

Exogenously, keratin materials are in the firing line for exposure to environmental factors which take part in their ageing, such as, for example, sunlight, especially via ultraviolet radiation, or else pollution.

The extrinsic ageing of the skin influenced by the environment supplements the intrinsic ageing linked to age, giving rise to characteristic skin damage and changes which are well described in the literature.

From a cellular and molecular perspective, skin ageing is reflected by a loss in the ability of keratinocytes and fibroblasts to proliferate; a reduction in differentiation markers such as, for example, transglutaminases (TGMs), and in particular transglutaminase 3; an imbalance between the production of the constituents of the dermis, such as, for example, collagen and fibrillin, and their destruction by different metalloproteases (MMPs), causing the scales to tip in the favor of degradation of the extracellular matrix and tissue destruction; the loss of molecules whose function is to participate in junctions and in anchoring the epidermis to the dermis, such as, for example, fibronectin and integrin, which leads to weakening of the dermoepidermal junction.

From a macroscopic perspective, skin ageing is apparent with the appearance of wrinkles and of slackness. The aged skin is finer and more fragile than a young skin. Finally, aged skin tends to lose its elasticity, its barrier function against external attacks, and its ability for repair.

Several compositions or methods for preventing and/or treating the signs of skin ageing are known in the prior art.

For example, use of a non-photosynthetic, non-fruiting filamentous bacterial extract has been proposed (FR 2 719 768 and FR 2 838 056). In this case, prevention and/or treatment of the signs of skin ageing is/are especially obtained with an extract of Vitreoscilla filiformis containing lipopolysaccharides, by inducing an increase in the endogenous synthesis of superoxide dismutase (FR 2 838 056).

Moreover, it has been described in document FR 2 762 782 that a clarified and stabilized culture medium of at least one non-photosynthetic filamentous bacterium has beneficial properties from a perspective of reducing and/or delaying skin ageing.

There is therefore a need for novel active agents or novel treatments for preventing and/or treating skin damage, and more particularly in aged skin.

There is a need for novel anti-ageing cosmetic treatments, able to reactivate epidermal regeneration by inducing keratinocyte proliferation.

There is a need for novel anti-ageing cosmetic treatments, able to maintain, or even reinforce, the skin's barrier properties, by repairing skin lesions.

### SUMMARY OF THE INVENTION

According to a first aspect, the invention relates to a carbohydrate fraction isolated from a clarified lysate obtained by thermolysis and proteolysis of a biomass of a bacteria belonging to the genus Vitreoscilla sp.

According to another aspect, the invention relates to a composition comprising, in a physiologically acceptable medium, a carbohydrate fraction according to the invention. The composition is advantageously a cosmetic or dermatological composition.

According to another aspect, the invention relates to the use of a carbohydrate fraction according to the invention, as cosmetic and/or dermatological active agent, preferably as anti-ageing active agent.

According to another aspect, the invention relates to a cosmetic process for treating a keratin material, comprising a step of topical application of a carbohydrate fraction or of a composition according to the invention.

According to another aspect, the invention relates to cosmetic process for treating and/or preventing the signs of ageing of the skin, comprising a step of topical application of a carbohydrate fraction or of a composition according to the invention.

According to another aspect, the invention relates to a process for preparing a carbohydrate fraction, isolated from a lysate of a biomass of a bacteria belonging to the genus Vitreoscilla sp., comprising the steps consisting in:
providing a biomass of a bacterium or bacteria belonging to the genus Vitreoscilla sp.;
thermolyzing and proteolyzing said biomass, in order to obtain a lysate;
clarifying the lysate, in order to obtain a clarified lysate;
purifying the polysaccharides of the clarified lysate.

According to another aspect, the invention relates to a carbohydrate fraction isolated from a clarified lysate of a biomass of bacteria belonging to the genus Vitreoscilla sp., wherein said carbohydrate fraction is obtained with a process comprising the following steps:

providing a biomass of bacteria belonging to the genus Vitreoscilla sp.;

thermolyzing and proteolyzing said biomass, in order to obtain a lysate;

clarifying the lysate, in order to obtain a clarified lysate; and

purifying the polysaccharides of the clarified lysate.

### DETAILED DESCRIPTION OF THE INVENTION

The inventors have observed, surprisingly, that a carbohydrate fraction isolated from a lysate of a biomass of bacteria belonging to the genus Vitreoscilla sp. has beneficial properties from the perspective of caring for the skin, the mucous membranes, the scalp and/or the keratin materials.

Indeed, after application of the carbohydrate fraction to the keratin materials, it forms a deposit on the keratin materials which has cosmetic benefits.

In particular, the carbohydrate fraction applied to the hair gives them a soft and smooth feel and good disentangling.

When applied onto the epidermis, the carbohydrate fraction stimulates the expression of the gene TGM3, encoding for the transglutaminase 3.

“Carbohydrate fraction” means a fraction consisting of at least 95% monosaccharides and/or polysaccharides, preferably polysaccharides.

Thus, a carbohydrate fraction within the meaning of the invention comprises between 0% and at most 5% amino acids, peptides, proteins, fatty acids, lipids, nucleotides, nucleosides, nucleic acids, synthesis intermediates, metabolic intermediates, and mixtures thereof.

It is recalled that the use of a total lysate of bacteria belonging to the genus Vitreoscilla sp., in a complete fermentation medium, is known from the prior art as an active agent for preventing and/or treating hyperseborrheic conditions not associated with a dandruff condition of the scalp (FR 2 988 607) or else as an active agent for preventing and/or treating dandruff conditions of the scalp, especially associated with a proliferation of pathogenic microorganisms on the scalp and/or an imbalance in the scalp ecoflora (FR 2 973 700).

The lysates described in these documents thus comprise a mixture of protein, lipid and polysaccharide fractions and are not intended for treating and/or preventing skin ageing.

Moreover, the carbohydrate fraction according to the invention is distinguished from the lipopolysaccharide extract described by the document FR 2 838 056, especially by its composition, almost exclusively consisting of polysaccharides. It is recalled that the lipopolysaccharide described by FR 2 838 056 has a characteristic ternary structure, consisting of a lipid A, a central (core) oligosaccharide, and saccharide side chains, the latter constituting the O antigen.

Isolated Carbohydrate Fraction According to the Invention

According to a first aspect, the invention relates to a carbohydrate fraction isolated from a clarified lysate obtained by thermolysis and proteolysis of a biomass of bacteria belonging to the genus Vitreoscilla sp.

“Carbohydrate fraction” means the polysaccharides initially contained in the bacterial lysate.

“Polysaccharide” means a chain of at least two monosaccharides bonded to one another by glycosidic bonds.

The polysaccharides may have a linear or branched structure.

The polysaccharides especially comprise subunits comprising between 5 and 6 carbon atoms, such as subunits chosen from a group comprising mannose, rhamnose, glucose, galactose, arabinose and fructose.

The polysaccharides have a molecular weight of between 5 kDa and 200 kDa.

In one particular embodiment, the polysaccharides have a molecular weight of between approximately 10 kDa and approximately 150 kDa.

In certain embodiments, the carbohydrate fraction corresponds to the total carbohydrate fraction of said bacterial lysate, that is to say comprises the bacterial cell membrane polysaccharides, the intracellular polysaccharides, and the polysaccharides dissolved in the aqueous fraction of the bacterial culture medium, namely the complete fermentation medium.

In certain embodiments, the carbohydrate fraction essentially comprises, or alternatively exclusively comprises, the bacterial cell membrane polysaccharides, the intracellular polysaccharides or the polysaccharides dissolved in the aqueous fraction of the complete fermentation medium.

In certain embodiments, the carbohydrate fraction essentially comprises, or alternatively exclusively comprises, a mixture of bacterial cell membrane polysaccharides and intracellular polysaccharides.

In certain embodiments, the carbohydrate fraction corresponds to a mixture of bacterial cell membrane polysaccharides and polysaccharides dissolved in the aqueous fraction of the complete fermentation medium.

In certain embodiments, the carbohydrate fraction essentially comprises, or alternatively exclusively comprises, a mixture of intracellular polysaccharides and polysaccharides dissolved in the aqueous fraction of the complete fermentation medium.

In one particular embodiment, the carbohydrate fraction comprises polysaccharides chosen from a group comprising polysaccharides dissolved in the complete fermentation medium and a mixture of intracellular polysaccharides and bacterial cell membrane polysaccharides.

Within the context of the invention, “isolated” means the result of the physical separation of the carbohydrate fraction within the meaning of the invention from the other fractions contained in the lysate after thermolysis and proteolysis, namely the result of the physical separation of the other fractions encompassing the peptide/protein fraction, the lipid fraction, the nucleic acids fraction and the fraction containing the metabolites originating from the bacterial culture, such as unconsumed amino acids and synthesis intermediate compounds used in cellular metabolic pathways.

In one particular embodiment, the carbohydrate fraction is devoid of amino acids, peptides, proteins, fatty acids, lipids, nucleotides, nucelosides, nucleic acids, synthesis intermediates, metabolic intermediates, and mixtures thereof.

Bacteria Belonging to the Genus Vitreoscilla sp (Especially Species: Vitreoscilla filiformis)

As specified above, the microorganism considered according to the invention in the lysate state is a non-photosynthetic filamentous bacterium as defined according to the classification in Bergey's Manual of Systematic Bacteriology (Vol. 3, sections 22 and 23, 9^{th} edition, 1989), and belonging to the genus Vitreoscilla sp. (especially species: Vitreoscilla filiformis).

As will emerge from the examples presented below, the inventors have discovered, unexpectedly, that the carbohydrate fraction of a lysate of a biomass of such a bacterium, optionally formulated in a complete fermentation medium, has unexpected cosmetological and/or dermatological properties.

More particularly, it is a bacterium belonging to the genus Beggiatoa, Vitreoscilla, Flexithrix or Leucothrix.

According to a preferred variant of the invention, it is the bacterium Vitreoscilla filiformis.

In one particular embodiment, the bacterium is the Vitreoscilla filiformis strain, preferably the Vitreoscilla filiformis strain ATCC 15551.

Complete Fermentation Medium

In one particular embodiment, the lysate of bacteria belonging to the genus Vitreoscilla sp. also comprises a complete fermentation medium.

Within the meaning of the present invention, the expression “lysate in a complete fermentation medium” means that the lysate is formulated in the complete culture medium in which the bacteria were cultured until after the microbial growth phase which led to the use of the nutritive substrates initially present in the culture medium.

Within the meaning of the present invention, the expression “complete fermentation medium” is intended to denote a medium originating from a culture method which has been used for the growth and cell lysis of the microorganism, said medium moreover not having been subjected to any additional handling with the aim of separating and/or eliminating all or some of the non-aqueous constituents thereof.

More particularly, the active agent considered according to the invention is formed of the carbohydrate fraction of a lysate of microorganisms and of all or some, in terms of amount, of the culture medium having served for the fermentation of said bacterium and in which the cell lysis thereof was consecutively carried out (i.e. complete fermentation medium).

Consequently, an active agent according to the invention, that is to say an active agent obtained from a carbohydrate fraction of a lysate of bacteria belonging to the genus Vitreoscilla sp. (especially species: Vitreoscilla filiformis), optionally in a complete fermentation medium, is clearly distinct from a total lysate or a supernatant of a fermentation medium for a bacterium belonging to the genus Vitreoscilla sp. (especially species: Vitreoscilla filiformis).

Moreover, the active agent considered according to the invention is distinct from a peptide/protein fraction, from a lipid fraction, from a nucleic acid fraction, and from mixtures thereof, which would be isolated or obtained from a lysate of a biomass of bacteria belonging to the genus Vitreoscilla sp. (especially species: Vitreoscilla filiformis), optionally in a complete fermentation medium.

Indeed, the active agent considered according to the invention, in opposition to this biomass or a biomass fraction other than the carbohydrate fraction, this lysate or lysate fraction other than the carbohydrate fraction, contains a significant amount of water-soluble cellular polysaccharides, that is to say contained in said bacterium, or water-soluble extracellular polysaccharides, which polysaccharides are released naturally into the culture medium during the proliferation of said bacteria.

Thus, the expression “complete medium” also extends to a form of the complete medium which is “concentrated” in terms of the fact that it is obtained following a partial evaporation of the water constituting a fermentation medium in which, consecutively, the culture of the corresponding microorganism and the cell lysis thereof have been carried out. Of course, this evaporation is carried out under operating conditions adjusted so as not to adversely affect the integrity of the non-aqueous constituents forming this complete medium.

Within the meaning of the present invention, the expression “non-aqueous constituents” implies that water, which is a predominant constituent in conventional fermentation media, is not one of the constituents which has to remain as is, that is to say at equal amounts, in the complete culture medium according to the invention.

Composition of Fermentation Medium

By definition, a fermentation or else culture medium is a support which enables the culture, and hence depending on the case, the growth, of cells, of bacteria or of yeasts. In principle, the cells find, in this medium, the essential components for their rapid multiplication in large numbers, and also sometimes elements which will enable the growth of a specific genus of bacteria or of a particular family to be favored, in this instance a bacterium belonging to the genus Vitreoscilla sp. (especially, species: Vitreoscilla filiformis).

The composition thereof must therefore satisfy the nutritional requirements of the microorganism considered and which are necessary for the proliferation of the latter.

More specifically, the composition of this culture medium must:
serve the requirements for mineral ions and growth factors and provide the source of carbon and energy;
have a pH close to the optimal pH; and
have an optimal ionic strength (the medium may be isotonic, but this is not compulsory).

Thus, the composition of a fermentation medium suitable for the invention comprises at least:
a source of carbon and energy, generally represented by a sugar, and advantageously glucose;
a source of potassium and phosphorus, just like, for example, K₂HPO₄;
a source of nitrogen and sulfur, which may be represented by the compound (NH₄)₂SO₄;
a source of magnesium, such as, for example, MgCl₂;
a source of calcium, such as, for example, CaCl₂;
a source of iron, and more particularly iron citrate, the citrate having the role of keeping the iron in solution;
a source of trace elements, chosen especially from Cu, Zn, Co, Ni, B or Ti salts;
a source of water, generally sterile, essential for all lifeforms; and
a pH buffer which may be represented by KH₂PO₄.

If one of these components is not present, the bacteria do not grow, since they cannot themselves compensate for the absence thereof.

By way of illustration of a fermentation medium suitable to the growth of a microorganism in accordance with the invention, mention may especially be made of the medium represented in example 1 below.

An effective amount of the microorganism considered according to the invention is introduced therein and everything is placed under conditions conducive to the proliferation thereof

Lysis of the Bacteria to Obtain an Active Agent According to the Invention

A lysate commonly denotes a material obtained following the destruction or dissolution of biological cells (biomass) via a phenomenon of “cell lysis”, thus causing the release of the intracellular and cellular biological constituents naturally contained in the biological cells considered.

Within the meaning of the present invention, the term “lysate” denotes all the intracellular and extracellular material, obtained by lysis of the microorganism concerned (biomass), namely a bacterium belonging to the genus Vitreoscilla sp. (especially species: Vitreoscilla filiformis).

In other words, the lysate used is thus formed from all the intracellular biological constituents thereof, especially the metabolites thereof and the cell wall and cell membrane constituents generated during the cell lysis thereof.

Within the meaning of the invention, the term “metabolite” denotes any substance originating from the metabolism of the microorganism considered according to the invention.

Consequently, a lysate considered according to the invention may be obtained via a process consisting of:
the culture of at least one bacterium belonging to the genus Vitreoscilla sp. (especially species: Vitreoscilla filiformis) on a fermentation medium under conditions conducive to the proliferation of said bacterium, and
the cell lysis of said bacteria in said fermentation medium.

The carbohydrate fraction within the meaning of the invention denotes the polysaccharides originating from the thermolysis and proteolysis of said biomass.

To obtain a lysate of a biomass of bacteria belonging to the genus Vitreoscilla sp. in a complete fermentation medium, according to a process comprising a step of culturing said bacteria, those skilled in the art may especially refer to example 1.

Advantageously, to obtain a lysate of bacteria belonging to the genus Vitreoscilla sp. in a complete fermentation medium, the biomass (bacterial cells after the growth phase, present in the medium in which they were cultured) may be previously frozen, for example at a temperature of −20° C., then is sterilized, preferably by heat, in particular by subjecting the previously frozen biomass to a step of heating at a temperature of greater than 100° C. By way of illustration, the step of sterilizing the biomass may be carried out by autoclaving, for example at a temperature of 121° C.

As emerges from the preceding text, following this culture of said microorganism, the latter is transformed into the lysate state directly within the fermentation medium which served for the culture thereof.

In one particular embodiment, said lysate is obtained by a step of thermolysis and a step of proteolysis of said biomass.

In one particular embodiment, the proteolysis precedes the thermolysis.

In a preferred embodiment, the thermolysis precedes the proteolysis.

Composition

The carbohydrate fraction according to the invention is easy to formulate within a composition which comprises at least one ingredient conventionally used in cosmetics and/or dermatology.

This is because it is odorless, and therefore does not require the concomitant presence of fragrance, especially in order to mask an unpleasant odor.

Moreover, the carbohydrate fraction according to the invention is colorless, and therefore does not cause any untimely or undesired coloration once it has been introduced into a cosmetic and/or dermatological composition.

Thus, the invention also relates to a composition, especially cosmetic and/or dermatological, comprising, in a physiologically acceptable medium, a carbohydrate fraction as described by the present invention.

The term “physiologically acceptable medium” means a medium that is compatible with human keratin materials, in particular with the skin and the hair.

In one particular embodiment, the cosmetic and/or dermatological composition comprises a carbohydrate fraction isolated from a clarified lysate obtained by thermolysis and proteolysis of a biomass of bacteria belonging to the genus Vitreoscilla sp., which lysate also comprises a complete fermentation medium.

A composition containing the active agent according to the invention may be applied topically.

A composition according to the invention is therefore applied for purely esthetic purposes, that is to say in order to improve the appearance and the texture of the skin, of a mucous membrane, of the scalp and of the keratin materials. In other words, within the meaning of the invention, the term “apply” does not cover a treatment for medical purposes.

A composition according to the invention advantageously comprises an amount of carbohydrate fraction isolated from a clarified lysate obtained by thermolysis and proteolysis of a biomass of bacteria belonging to the genus Vitreoscilla sp., optionally in a complete fermentation medium, ranging from 0.1% to 10% by weight relative to the total weight of dry extract of said composition.

In certain embodiments, a composition according to the invention advantageously comprises an amount of carbohydrate fraction ranging from 0.5% to 5% by weight relative to the total weight of dry extract of said composition.

In other embodiments, a composition according to the invention advantageously comprises an amount of carbohydrate fraction ranging from 1% to 5% by weight relative to the total weight of dry extract of said composition.

A composition according to the invention may therefore be in any galenical form normally available for the specific selected mode of administration.

The support may be of diverse nature according to the type of composition considered.

As more particularly regards compositions intended for external topical administration, they may be aqueous, aqueous-alcoholic or oily solutions, solutions or dispersions of the lotion or serum type, emulsions of liquid or semi-liquid consistency of the milk type, obtained by dispersing a fatty phase in an aqueous phase (O/W) or vice-versa (W/O), or suspensions or emulsions, of soft, semi-solid or solid consistency, of the cream type, aqueous or anhydrous gel, microemulsions, microcapsules, microparticles, or vesicular dispersions of ionic and/or nonionic type.

These compositions are prepared according to the usual methods.

These compositions may especially constitute cleansing, protective, treatment or care creams, lotions, gels or mousses for caring for the skin, the mucous membranes, the scalp and/or the keratin materials.

They may be used for the cosmetic and/or dermatological treatment of the skin, the mucous membranes, the scalp and/or the keratin materials, in the form of solutions, creams, gels, emulsions, mousses or else in the form of compositions adapted for use in an aerosol, for example also containing a pressurized propellant.

In certain embodiments, a topical composition according to the invention, may advantageously be formulated in any galenical form that is suitable for haircare, especially in the form of a hair lotion, a shampoo, a conditioner, a detangler, a hair cream or gel, a styling lacquer, a hair-setting lotion, a treating lotion, a dye composition (especially for oxidation dyeing) optionally in the form of a coloring shampoo, a hair-restructuring lotion, a permanent-waving composition, a lotion or gel for combating hair loss, an antiparasitic shampoo or a medicated shampoo, especially an anti-seborrhoea shampoo, a scalp care product, which is especially anti-irritant, anti-ageing or restructuring, or which activates the blood circulation.

In a known manner, the galenical forms intended for topical administration may also contain adjuvants that are common in the cosmetic and/or dermatological field, such as thickeners, oils, waxes, preserving agents, antioxidants, solvents, fragrances, fillers, UV screening agents and dyestuffs.

The amounts of these various adjuvants are those conventionally used in the field under consideration, for example from 0.01% to 20% of the total weight of the composition. Depending on their nature, these adjuvants may be introduced into the fatty phase and/or into the aqueous phase.

The composition of the invention may also advantageously contain water. The water may be a spring and/or mineral water, chosen in particular from Vittel water, waters from the Vichy basin and la Roche Posay water.

The water may be present in a content ranging from 5 to 99% by weight, preferably ranging from 20 to 95% by weight and preferentially ranging from 40% to 95% by weight, relative to the total weight of the composition.

Use and Processes

Moreover, the invention relates to the use of a carbohydrate fraction able to be obtained by the process of the invention, as cosmetic and/or dermatological active agent, preferably as anti-ageing active agent.

“Cosmetic and/or dermatological active agent” means an active agent having beneficial properties after application to keratin materials.

A beneficial property might be, for example, the stimulation of the expression of proteins that are present in young skin, and have a reduced expression in aged skins, such as transglutaminases.

The invention also relates to a non-therapeutic cosmetic process for treating a keratin material, comprising a step of topical application, to the keratin material, of a carbohydrate fraction isolated from a lysate of a biomass of bacteria belonging to the genus Vitreoscilla sp. or of a composition comprising such a fraction, it being understood that said carbohydrate fraction is obtained after clarification of a lysate obtained by thermolysis and proteolysis of a biomass of bacteria belonging to the genus Vitreoscilla sp.

Within the context of the invention, “treating a keratin material” means the topical application of a carbohydrate fraction according to the invention for caring for the skin, the mucous membranes, the lips, the nails, the scalp and the hair.

The invention also relates to a non-therapeutic cosmetic process for treating and/or preventing the signs of skin ageing, comprising a step of topical application, to the skin, of a carbohydrate fraction isolated from a lysate of a biomass of a bacterium or bacteria belonging to the genus Vitreoscilla sp. or of a composition comprising such a fraction, it being understood that said carbohydrate fraction is obtained after clarification of a lysate obtained by thermolysis and proteolysis of a biomass of bacteria belonging to the genus Vitreoscilla sp.

Within the context of the present invention, “treating the signs of skin ageing” means the reduction or easing of wrinkles, fine wrinkles, withering of the skin, loss of skin elasticity, loss of skin tonicity, thinning of the dermis, degradation of collagen fibers, loss of skin firmness, skin dryness.

Within the context of the present invention, “preventing the signs of skin ageing” means the absence of appearance, or the delayed appearance, of wrinkles, fine wrinkles, withering of the skin, loss of skin elasticity, loss of skin tonicity, thinning of the dermis, degradation of collagen fibers, loss of skin firmness, skin dryness.

In one particular embodiment, the lysate of a biomass of bacteria belonging to the genus Vitreoscilla sp. also comprises a complete fermentation medium.

The invention also relates to a process for preparing a carbohydrate fraction isolated from a lysate of a biomass of bacteria belonging to the genus Vitreoscilla sp., comprising the steps consisting in:
providing a biomass of bacteria belonging to the genus Vitreoscilla sp.;
thermolyzing and proteolyzing said biomass, in order to obtain a lysate;
clarifying the lysate, in order to obtain a clarified lysate;
purifying the polysaccharides of the clarified lysate.

“Biomass” means all or some of the mass of living or dead organisms contained in a completed culture, that is to say after disappearance of all or some of the nutritive elements constituting the initial complete fermentation medium.

In a preferred embodiment, the thermolysis precedes the proteolysis.

In one particular embodiment, the lysate of a biomass of bacteria belonging to the genus Vitreoscilla sp. also comprises a complete fermentation medium.

According to one particular embodiment, the thermolysis may be carried out at a temperature of between 60° C. and 120° C., preferably a temperature of between 70° C. and 100° C., preferably a temperature of approximately 80° C.

Approximately 80° C. means a temperature of 80° C.±5° C.

According to one particular embodiment, the thermolysis may be carried out for a duration of between 30 min and 240 min, preferably a duration of between 45 min and 90 min, preferably a duration of approximately 60 min.

Approximately 60 min means a duration of 60 min±5 min.

According to one particular embodiment, the proteolysis may be carried out by any proteolytic enzyme known in the prior art, among which mention may be made of chymotrypsin, trypsin, papain, pepsin, subtilisin and mixtures thereof.

In one particular embodiment, the proteolysis may be carried out by an alcalase, in particular by the Alcalase® 2.4 L FG (subtilisin sold by Novozyme).

In a preferred embodiment, the proteolysis is not carried out by proteinase K.

When the proteolysis is carried out by means of a commercially available proteolytic enzyme, reference will be made to the manufacturer's instructions as regards the amount of protease to use, the duration and the temperature of incubation of the lysate with the protease and the pH of the reaction medium.

“Reaction medium” means the aqueous solution comprising the lysate of bacteria belonging to the genus Vitreoscilla sp., optionally in a complete fermentation medium, the protease, any other ingredient optionally necessary for the proteolysis, for example a buffer enabling the adjustment of the concentrations of salts and the adjustment of the pH.

It is within the ability of those skilled in the art to determine the optimal conditions for carrying out the proteolysis of the lysate, optionally in its complete fermentation medium.

In general, the proteolysis may be carried out with an amount of enzyme in the reaction medium of between 0.005% and 5% by weight, preferably between 0.05% and 1.5% by weight, relative to the total weight of the reaction medium.

Moreover, the duration of the proteolysis is between 30 min and 24 h, as a function of the amount of protease used and the protein content of the initial medium.

In one particular embodiment, the reaction medium is kept at a constant temperature of between 20° C. and 80° C., preferably a temperature of between 30° C. and 60° C.

In one particular embodiment, the pH of the reaction medium is between 3 and 10, preferably between 6 and 9.5, preferably around 8.

In one particular embodiment, when the proteolysis is carried out in the presence of subtilisin, the pH of the reaction medium is around 7.5.

Moreover, the process for preparing the lipopolysaccharide using the proteinase K as described in the document FR 2 838 056 differs from the process for preparing a carbohydrate fraction as described by the invention in that the thermolysis step is followed by a centrifugation step, the supernatant then being subjected to the step of proteolysis by means of the proteinase K.

Unlike the process described by the document FR 2 838 056, it is important that the thermolysis and the proteolysis, as carried out according to the invention, are conducted in the same reaction medium, in order to retain all the carbohydrate fractions contained in the lysate, and concomitantly reduce the number of handling operations of the polysaccharides and hence do away with loss thereof during the various purification steps.

According to one particular embodiment, the proteolysis may be followed by a proteolysis inactivation step, for example by means of an inhibitor of the protease used or a cocktail of inhibitors; by incubation of the reaction medium at a temperature of between 70° C. and 90° C., for example at a temperature of approximately 85° C.; by a variation of the pH relative to the pH of the reaction medium.

Without wishing to be bound by any theory, the inventors consider that the thermolysis and the proteolysis of the lysate lead(s) to eliminating the enzymes intrinsic to the bacterial strain which could make a negative contribution to the cohesion of the skin and the dermoepidermal junction.

In one particular embodiment, the proteolysis inactivation step is carried out for a duration ranging from a few minutes to several hours, for example a duration between 5 min and 2 h, preferably a duration of between 10 min and 30 min.

The conditions of the proteolysis inactivation step may be adapted by those skilled in the art.

In certain particular embodiments, the lysate does not comprise denaturing and/or chaotropic agents.

Within the context of the present invention, “denaturing and/or chaotropic agent” means an agent capable, under standard conditions, of unfolding the proteins contained in the lysate, that is to say of especially doing away with all or some of the secondary or tertiary structures, which results in an unfolded and substantially linear protein structure.

According to one particular embodiment, the process for preparing a carbohydrate fraction according to the invention may also comprise a step of acidifying the medium, in general after the step of thermolysis and/or proteolysis of the biomass. Any acid suited to this step may be used, such as, for example, an acid selected from a group comprising citric acid, hydrochloric acid, sulfuric acid, phosphoric acid and mixtures thereof.

According to one particular embodiment, the process for preparing a carbohydrate fraction according to the invention may also comprise a step of clarifying the optionally acidified lysate, for example by a succession of depth filtrations, by means of suitable filters, for example filters having pores with a diameter of between approximately 0.4 μm and 20 μm, which includes filters having retention ranges of approximately 0.4-0.8 μm, 1-4 μm, 4-7 μm, 7-15 μm and 8-20 μm.

Implementing the invention comprises a step of purifying the polysaccharides, which may be carried out by any method known from the prior art (Gonçalves et al. 2007; Communicating Current Research and Educational Topics and Trends in Applied Microbiology; Mendez-Vilas Ed.).

For example, the polysaccharides may especially be purified by alcohol precipitation, especially by ethanol precipitation; more preferentially by separation by membrane filtration.

In one particular embodiment, the process for preparing a carbohydrate fraction according to the invention may also comprise a step of concentrating the polysaccharides by membrane ultrafiltration, followed by purification by membrane diafiltration. It is known from the prior art that the diafiltration volume determines the desired purity grade.

In this particular embodiment, the process may employ a step of membrane ultrafiltration, especially by means of filters having suitable cut-off thresholds.

In practice, suitable filters are for example filters having thresholds of 30 000 Da, 10 000 Da or 1000 Da.

In one particular embodiment, a filter suited to a membrane ultrafiltration is based on regenerated hydroxyethyl cellulose and polyether sulfone.

In one particular embodiment, a filter suited to a membrane ultrafiltration preferably has a cut-off threshold of 30 000 Da and is based on regenerated hydroxyethyl cellulose.

In this particular embodiment, a final polysaccharide concentration may be between 0.1% and 10% by weight, preferably between 0.5% and 5% by weight, preferably between 1% and 2% by weight, relative to the total weight of the fraction.

In one particular embodiment, one or more cosmetic preservative(s) routinely used in the field of cosmetics and/or dermatology, for example phenoxyethanol, may be added in order to reinforce the stability of the active agent during storage thereof.

### Examples

### 1—Preparation of a Carbohydrate Fraction within the Meaning of the Invention

The bacterial strain Vitreoscilla filiformis is cultured in a 3000 liter industrial fermenter. At the end of culture, the spontaneous pH is 6.2, the solids content is 0.6% and the free glucose content is 0.035%.

The strain V. filiformis is fermented in its complete culture medium, the composition of which is described in table 1 below.

TABLE 1
composition of the complete nutritive medium
for the culture of V. filiformis.

| Chemical name | [concentration] | |
|---|---|---|
| Autolytic yeast extract | 4 | g/l |
| Soybean papain peptone F | 3 | g/l |
| Glucose-roferose | 3 | g/l |
| KH₂PO₄ | 0.088 | g/l |
| CaCl₂ | 0.050 | g/l |
| CuSO₄•5H₂O | 60 | μg/l |
| MnSO₄•1H₂O | 152 | μg/l |
| KI | 20 | μg/l |
| ZnSO₄•7H₂O | 200 | μg/l |
| AlCl₃•6H₂O | 100 | μg/l |

| | |
|---|---|
| Osmosed water | qs 1 l |

To obtain a cosmetic active agent according to the invention, the following process is carried out.

The initial strain of V. filiformis is obtained from the ATCC (strain 15551).

The biomass is obtained by fermentation in an industrial bioreactor of 3000 effective liters; the composition of the culture medium is given above. During the culture, the pH is kept constant (7.00), as is the temperature (26° C.) and the dissolved oxygen (0.5). The culture is stopped when the solids content reaches 0.6% and the glucose content reaches 0.035%.

For extraction of the polysaccharides from the culture, a cycle of thermolysis is first applied for a duration of 15 min at a temperature of 80° C.

After cooling the medium to a temperature of 55° C., the pH is adjusted and kept at pH=8 by addition of a 1 M aqueous solution of sodium hydroxide. Proteolysis is then carried out by adding 0.1% (w/w) of protease isolated from Bacillus liceniformis: Alcalase® 2.4 L FG (from Novozyme®).

At the end of 3 hours of proteolysis, the medium is neutralized to pH 7 by means of an aqueous solution of phosphoric acid at 20% by weight. The enzyme is inactivated by heating the medium at a temperature of 90° C. for 10 minutes.

In order to avoid a blockage, the medium is clarified on regenerated cellulose membranes with porosity of K700, K100, EKS and 0.45+0.2 μm (from Pall).

A concentration step is then carried out on a regenerated cellulose cassette with a cut-off threshold of 30 kDa (from Pall). 1.5% phenoxyethanol is finally added to the retentate obtained after concentration.

### 2—Activity of a Carbohydrate Fraction According to the Invention in Sensory Terms

The active agent at 5% in water, on a lock of hair, showed positive activity in terms of the sensory nature of the fibers.

20 ml of a solution of active agent is prepared by diluting the active agent obtained in example 1 in municipal water.

Lock Preparation

1 standard lock of 2.7 g, of SA11.2 quality, was divided in three lots of 0.9 g equivalent weight.

Treatment of the Locks

Control lock: the control lock was soaked in 10 ml of municipal water for 5 min, with magnetic stirring at a rate of 400 rpm. 3 successive washing operations with municipal water were then carried out, with manual wringing out between each washing operation. At the end of the last washing operation, the lock was dried with a hairdryer.

2 Treated locks: the locks were soaked in 10 ml of solution of active agent for 5 min, with magnetic stirring at a rate of 400 rpm. The locks treated with the active agent were then handled in the same way as the control lock (washing and drying).

Sensory Assessment

Sensory assessment was carried out by a panel of 4 people. Each person touches the locks by sliding the lock between the thumb and the index finger.

Results

The two locks which were treated with the active agent at 5% had a much softer and smoother feel compared to the lock treated with municipal water. Moreover, the two treated locks disentangled much more easily.

### 3—Use of a Carbohydrate Fraction According to the Invention for Inducing the Expression of TCM3 Gene (Coding for Transglutamine 3) on a Model of Reconstructed Epidermis

### Biological Model:

Reconstructed epidermis models are the Skinethic™ RHE provided by Skinethic™ laboratories (Lyon, France) at Day 17; their quality was validated by histological control.
Culture conditions: All the experiments were carried out in a humid atmosphere at 37° C., 5% CO 2

### Preparation of Samples:

Untreated control group: epidermis are incubated on a control medium, without any compound of the invention;
Tested group: epidermis are incubated with the compound of the invention: solutions were prepared at final concentrations of 0.001 mg/ml and 0.0001 mg/ml of active agent in the control medium.

### Treatments:

After an overnight stabilization in a maintenance medium at 37° C., 5% CO 2, the initial medium is replaced with a culture medium containing, or not, the compound of the invention, for 18 hours at 37° C., under 5% CO2.
Three different non-cytotoxic concentrations of the compound were evaluated. Each dose was tested in duplicate.
In order to detect and demonstrate an anti-aging activity of the compound of the invention, reference compounds known for having anti-aging properties were evaluated in the same assay.

### Quantitative RT-PCR:

At the end of the treatment, the culture media were eliminated and the epidermis were rinsed 2 times in PBS buffer, and then frozen at −80° C. in RNAlater®.
After extraction with the Qiagen® Rneasy 96 Universal Tissue Kit, the RNAs were purified by DNase treatment and eluted in 90 μl of distilled water RNase free.
The quantification of the RNAs was carried out by spectrometric assay at 260 nm (NanoDrop® ND-1000, NanoDrop Technologies).
The quality of the purified fractions was then checked (ratio A260/A280) with the Agilent Bioanalyzer 2100 using the Pico LabChip (Agilent Technologies) RNA 6000 kits.

### Inverse Transcription and Amplification of RNAs

Reverse transcription of RNA to cDNA was performed using the High Capacity cDNA Reverse Transcription R Kit (Applied Biosystems). A step of amplifying the cDNA by TaqMan® PreAmp Master Mix (Applied Biosystems) was performed before PCR.

### Real-Time PCR

The study of gene expression was carried out using TaqMan Gene Expression Assay (Applied Biosystems).
For standardization, the following household genes were used: TBP, RPL13A, B2M and ATPSB

### Results

The table 2 below summarizes the induction factors of the expression of the gene of interest TGM3 coding for Transglutaminase3, by the carbohydrate fraction of the invention, depending on the concentration (mg/mL) in active agent.

TABLE 2

| | |
|---|---|
| Concentration of the | Compound of the invention |

| active agent (mg/mL) | 0.001 | 0.0001 |
|---|---|---|
| Induction factor of TGM3 | 2 | 2 |

An induction factor of the gene, greater than or equal to 2, is considered as representing a significant effect.

At a concentration of 0.0001 mg/ml of active agent, the compound of the invention presents a significant efficiency in the induction of the gene of interest TGM3.

The compound of the invention is therefore of interest for its use as a cosmetic and/or dermatological agent, in particular as an anti-ageing active agent.

1. A carbohydrate fraction isolated from a clarified lysate obtained by thermolysis and proteolysis of a biomass of bacteria belonging to the genus Vitreoscilla sp.

2. The carbohydrate fraction as claimed in claim 1, wherein the lysate of bacteria belonging to the genus Vitreoscilla sp also comprises a complete fermentation medium.

3. The carbohydrate fraction as claimed in claim 2, which comprises polysaccharides dissolved in the complete fermentation medium and a mixture of intracellular polysaccharides and bacterial cell membrane polysaccharides.

4. The carbohydrate fraction as claimed in claim 1, wherein said bacterium belonging to the genus Vitreoscilla sp. is the strain V. filiformis, preferably the strain V. filiformis ATCC 15551.

5. A composition comprising, in a physiologically acceptable medium, a carbohydrate fraction as claimed in claim 1.

6. The composition as claimed in claim 5, wherein the lysate of bacteria belonging to the genus Vitreoscilla sp also comprises a complete fermentation medium.

7. A cosmetic and/or dermatological process, comprising a step of topical application of a carbohydrate fraction as claimed in claim 1, as active agent.

8. A cosmetic process for treating a keratin material, comprising a step of topical application to the keratin material of a carbohydrate fraction as claimed in claim 1.

9. A cosmetic process for treating and/or preventing the signs of ageing of the skin, comprising a step of topical application to the skin of a carbohydrate fraction as claimed in claim 1.

10. A process for preparing a carbohydrate fraction isolated from a lysate of a biomass of bacteria belonging to the genus Vitreoscilla sp., comprising the steps consisting in:
providing a biomass of bacteria belonging to the genus Vitreoscilla sp.;
thermolyzing and proteolyzing said biomass, in order to obtain a lysate;
clarifying the lysate, in order to obtain a clarified lysate;
purifying the polysaccharides of the clarified lysate.

11. The process as claimed in claim 10, in which the thermolysis precedes the proteolysis.

12. The cosmetic and/or dermatological process as claimed in claim 7, wherein the carbohydrate fraction is administered as anti-ageing active agent.

13. A cosmetic and/or dermatological composition for treating a keratin material comprising the composition of claim 5.

14. A composition for treating and/or preventing the signs of ageing of the skin comprising the composition of claim 5.
